(19) United States
(12) Patent Application Publication
(10) Pub. No.: US 2011/0178168 A1
(43) Pub. Date: Jul. 21, 2011

(54) COUMARIN COMPOUNDS FOR THE TREATMENT OF CARDIOVASCULAR DISEASES AND A PROCESS FOR PREPARING THE SAME

(76) Inventors: Virender Singh Parmar, Delhi (IN); Hanumanthrao Guru Raj, Delhi (IN); Ashok Kumar Prasad, Delhi (IN); Subhash Chand Jain, Delhi (IN)

(21) Appl. No.: 13/001,031

(22) PCT Filed: Jun. 23, 2009
(86) PCT No.: PCT/IN09/00359

§ 371 Date: Apr. 11, 2011

(30) Foreign Application Priority Data
Jun. 23, 2008 (IN) 1495/DEL/2008

(51) Int. Cl. A61K 31/366; C07D 311/06; A61P 9/00; A61P 7/00

(52) U.S. Cl. 514/456; 549/289

(57) ABSTRACT

The present invention relates to compounds of Formula (I): wherein—X and Y represent O, S, NR′; Rₙ represents alkyl, aryl, OR₁, NH₂, SR₁, NR₁R₂. wherein R₁, R₂=H, alkyl, phenyl, aryl, OCOR₃, SCOR₃, NHCOR₃, NR₁COR₃, etc. (wherein R₃ represents alkyl, phenyl, aryl, heteroaryl); R′ and R″ represent H, alkyl, phenyl, substituted phenyl, phenyloxy, substituted phenyloxy, amino, mono substituted amino, disubstituted amino, aryl, heteroaryl, aryloxy, heteroaryloxy, alkoxy, thioalkyl, thioalkyloxy, halo and a process for preparing the same.

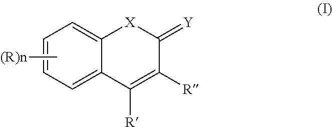

### TECHNICAL FIELD

This invention relates to coumarin compounds for the treatment of cardiovascular diseases and a process for preparing the same.

### BACKGROUND

Drugs that inhibit platelet function have assumed increasing importance in the care of patients with cardiovascular and cerebrovascular diseases, which are leading causes of death in the human population.

Physiological systems control fluidity of blood in mammals. Blood must remain fluid in the vascular systems and yet quickly be able to undergo hemostasis. Hemostasis or clotting begins when platelets first adhere to macromolecules in sub-endothelian regions of injured and/or damaged blood vessels. These platelets aggregate to form the primary haemostatic plug and stimulate local activation of plasma coagulation factors leading to generation of a fibrin clot that reinforces aggregated platelets.

Plasma coagulation factors, also referred to as protease zymogens, include factors II, V, VII, VIII, IX, X, XI, and XII. Coagulation or clotting occurs in two ways through different pathways. An intrinsic or contact pathway leads from XII to XIIa to XIa to IXa and to the conversion of X to Xa. Xa with factor Va converts prothrombin (II) to thrombin (IIa) leading to conversion of fibrinogen to fibrin. Polymerization of fibrin leads to a fibrin clot. An extrinsic pathway is initiated by the conversion of coagulation factor VII to VIIa by Xa. Factor VIIa, a plasma protease, is exposed to, and combines with its essential cofactor tissue factor (TF) which resides constitutively beneath the endothelium. The resulting factor VIIa/TF complex proteolytically activates its substrates, factors IX and X, triggering a cascade of reactions that leads to the generation of thrombin and a fibrin clot as described above.

While clotting as a result of an injury to a blood vessel is a critical physiological process for mammals, clotting can also lead to disease states. A pathological process called thrombosis results when platelet aggregation and/or a fibrin clot blocks (i.e., occludes) a blood vessel. Arterial thrombosis may result in ischemic necrosis of the tissue supplied by the artery. When thrombosis occurs in a coronary artery, a myocardial infarction or heart attack can result. A thrombosis occurring in a vein may cause tissues drained by the vein to become edematous and inflamed. Thrombosis of a deep vein may be complicated by a pulmonary embolism.

Preventing or treating clots in a blood vessel may be therapeutically useful by inhibiting formation of blood platelet aggregates, fibrin, thrombus formation, embolus formation, and for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels.

One drug to inhibit formation of blood platelet aggregation is the compound Aspirin. Aspirin inhibits platelet aggregation by irreversible inhibition of platelet cyclooxygenase and thus inhibits the generation of TXA2, a powerful inducer of platelet aggregation and vasoconstriction. Paradoxically, aspirin blocks synthesis of prostacyclin by endothelial cells, resulting in an effect that promotes platelet aggregation.

Clopidogrel hydrogen sulfate is a platelet aggregation inhibitor which was described for the first time in EP 281459. Clopidogrel is a potent, noncompetitive inhibitor of ADP-induced platelet aggregation (Plavix® PI). The active metabolite of clopidogrel binds to the low-affinity ADP-receptors. ADP binding to this site is necessary for activation of the GP IIb/IIIa receptor, which is the binding site for fibrinogen. Fibrinogen links different platelets together to form the platelet aggregate. Clopidogrel thus ultimately inhibits the activation of the GP IIb/IIIa receptor and it's binding with fibrinogen.

The present invention relates to novel coumarin compounds exhibiting antiplatelet activity. Coumarins are chemically known as benzopyrone compounds. They were first identified in 1820s, and exhibit a vanilla-like or freshly-mowed hay fragrance. The compound is generally found in many plants like Tonka beans, sweet clover grass, lavender and licorice. It is also present in fruit-bearing plants like apricots, cherries, strawberries, and cinnamon and dong quai. Artificial production of coumarin started since 1820s and has been used in the manufacture of flavorings and perfumes since 1868. Coumarin derivatives like warfarin have been known for their anticoagulant activity. Warfarin was first disclosed in U.S. Pat. No. 242,7578. Warfarin inhibits the vitamin K-dependent synthesis of biologically active forms of the calcium-dependent clotting factors II, VII, IX and X, as well as the regulatory factors protein C, protein S, and protein Z. Other proteins not involved in blood clotting, such as osteocalcin, or matrix Gla protein, may also be affected.

Warfarin does not exhibit platelet inhibitory activity but acts as an anticoagulant by competitive inhibitors of vitamin K in the biosynthesis of prothrombin. Certain coumarin derivates from Murraya omphalocarpa have shown antiplatelet activity.

The compounds in accordance with the present invention do not require metabolic activation like Clopidogrel. Moreover, Clopidogrel is reported to have an interaction with other drugs such as atrovastatin and exhibits inter individual variations. The compounds of the present invention were found to exhibit nearly equal antiplatelet activity when compared to clopidogrel at equimolar dose ex vivo. They are effective in causing the inhibition of ADP induced as well as collagen induced platelet aggregation both invitro and exvivo. One compound of the series has also been found to be more potent in inhibition of ADP induced platelet aggregation as compared to the other antiplatelet drugs like Aspirin exvivo. As effective antiplatelet agents, these compounds are expected to be useful in the treatment of cardiovascular diseases.

### OBJECTIVE

The principal object of the present invention is to provide coumarin compounds of Formula I which act as inhibitors of platelet aggregation.

Another object of the present invention is to provide coumarin compounds of Formula I which are cost effective and have a better efficacy.

Yet another object of the present invention is to provide coumarin compounds of Formula I which do not require metabolic activation.

### SUMMARY

The present invention relates to compounds of Formula I

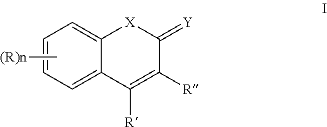

wherein—
X and Y represent O, S, NR′;
Rₙ represents alkyl, aryl, OR₁, NH₂, SR₁, NR₁R₂
wherein R₁, R₂=H, alkyl, phenyl, aryl, OCOR₃, SCOR₃, NHCOR₃, NR₁COR₃, etc. (wherein R₃ represents alkyl, phenyl, aryl, heteroaryl)
R′ and R″ represent H, alkyl, phenyl, substituted phenyl, phenyloxy, substituted phenyloxy, amino, monosubstitutedamino, disubstitutedamino, aryl, heteroaryl, aryloxy, heteroaryloxy, alkoxy, thioalkyl, thioalkyloxy, halo.

The present invention further relates to a process of preparing the compounds of Formula I comprising

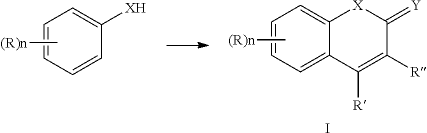

Wherein X, Y, Rₙ, R₁, R₂, R₃, R′ and R″ have the same meaning as stated above

### DESCRIPTION

The present invention relates to a compound of Formula I and a process for preparing the same.

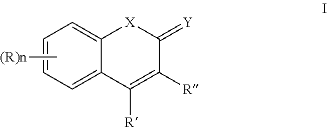

X and Y represent O, S, NR′;
Rₙ represents alkyl, aryl, OR₁, NH₂, SR₁, NR₁R₂
wherein R₁, R₂=H, alkyl, phenyl, aryl, OCOR₃, SCOR₃, NHCOR₃, NRiCOR₃, etc. (wherein R₃ represents alkyl, phenyl, aryl, heteroaryl)
R′ and R″ represent H, alkyl, phenyl, substituted phenyl, phenyloxy, substituted phenyloxy, amino, monosubstitutedamino, disubstitutedamino, aryl, heteroaryl, aryloxy, heteroaryloxy, alkoxy, thioalkyl, thioalkyloxy, halo.

The specific acyloxy groups attached with the coumarin compounds are attributed for enhancement of intracellular nitric oxide level leading to the inhibition of ADP induced platelet aggregation.

The present invention further relates to a process of preparing the compound of Formula I comprising
mixing polyphosphoric acid, a phenol and ethyl acetoacetate in the ratio 3.25:1:1 at 70-90° C. for 15-30 minutes to obtain a coumarin;
acylating said coumarin to obtain the product as claimed in claim I;
Purifying the product obtained.

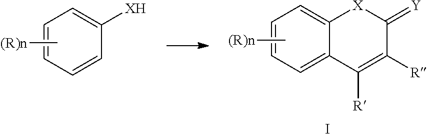

Wherein X, Y, Rₙ, R₁, R₂, R₃, R′ and R″ have the same meaning as stated above

The inventors of the present invention have found that certain coumarin compounds are very effective in the inhibition of ADP induced platelet aggregation as well as collagen induced platelet aggregation. Nitric oxide is known to mediate a number of pharmacological actions such as vasorelaxation, lowereing of blood pressure and inhibition of platelet aggregation. The compounds of the present invention have been found to enhance intracellular nitric oxide levels and hence act as antiplatelet agents. The specific acyl group attached to the coumarin derivative is attributed to the enhancement of intracellular nitric oxide level leading to the inhibition of ADP induced platelet aggregation.

The compounds of Formula I exhibit antiplatelet activity. Platelet Nitric Oxide Synthase (NOS) is remakably activated by way of acetylation catalyzed by Calreticulin transacetylase (CRTAase) leading to robust enhancement of NO in platelets. Compounds of Formula I enhance NO in platelets. Due to this activity, these compounds enhance the NOS leading to enhancement of platelet NO which contributes to the antiplatelet action. The compounds of the present invention were found to exhibit nearly equal antiplatelet activity when compared to clopidogrel at equimolar dose ex vivo. They are effective in causing the inhibition of ADP induced as well as collagen induced platelet aggregation both invitro and exvivo. These are also found to be more potent in inhibition of ADP induced platelet aggregation as compared to the other antiplatelet drugs like aspirin exvivo.

The compounds of the present invention can be synthesized in a two-step protocol using very inexpensive starting materials. Further the compounds do not contain any chiral center and thus special enantioselective synthesis or chiral resolution is not required for the preparation of these coumarin molecules as in the case of preparation of desired enantiomer of clopidogrel. Thus the cost of production of these compounds is much lesser than that of Clopidogrel.

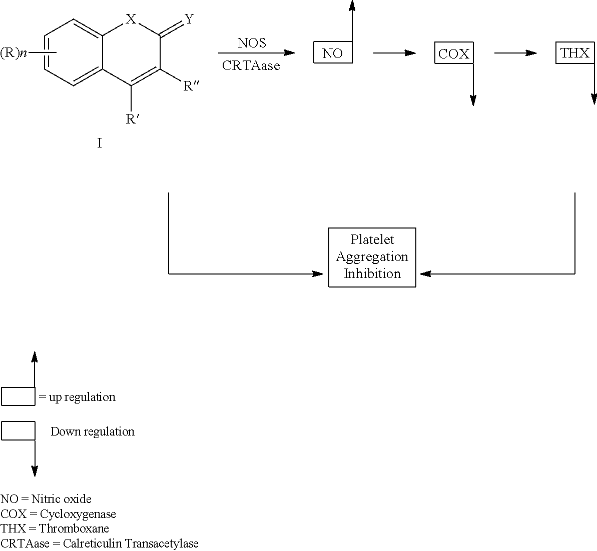

### Mechanism of Inhibition of Platelet Aggregation by Compounds of Formula I

Details of the study conducted are presented below.

A comparison of the efficacy of the compounds of the present invention with other known antiplatelet agents have been given in Table I.

TABLE I

| 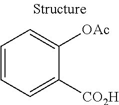 | TAase activity (Units) | ADP-induced platelet aggregation (% inhibition) IN VITRO | ADP-induced platelet aggregation (% inhibition) EX VIVO | Enhancement of nitric oxide (folds) |
|---|---|---|---|---|
| ASPIRIN | Nil | Nil | 55 | Nil |
| 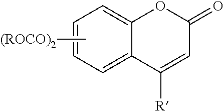 | 33 | 60 | 70 | 7 |
| 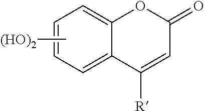 | 2 | 27 | — | Nil |
| 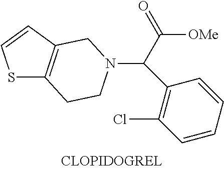 | Nil | Nil | 65 | Nil |

The present invention will now be described with the help of following examples.

### EXAMPLES

### 1. Synthesis of 4-methylcoumarin

### Reaction Involved:

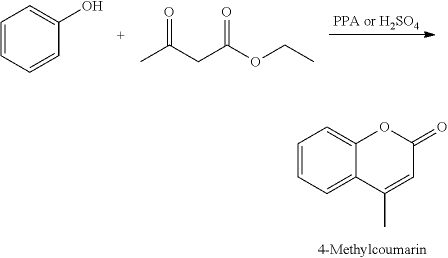

Procedure: A mixture of polyphosphoric acid (158 g, obtained by dissolving 125 g of phosphorous pentoxide in 72 g of commercial orthophosphoric acid, d. 1.75), phenol (9.4 g, 0.1 mol) and ethyl acetoacetate (13 g, 0.1 mol) was stirred and heated at 75-80° C. for 20 minutes. The reaction mixture was poured onto ice-water. The separated product was filtered, washed with cold water and crystallized from dilute alcohol. The pure compound 15.5 g (in 97% yield) was obtained.

### 2. Synthesis of 7-acetoxy-4-methylcoumarin

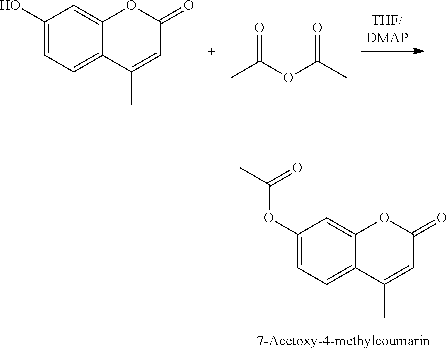

Procedure: To a mixture of 7-hydroxy-4-methylcoumarin (17.6 g, 0.1 mol) in CH₂Cl₂ (100 ml), was added acetic anhydride (10.3 ml, 0.11 mol), followed by catalytic amount of DMAP (0.122 g, 0.01 mol). Reaction mixture was stirred at room temperature for 2-3 hrs and reaction completion was monitored by TLC. After reaction completion, CH₂Cl₂ was evaporated on a rota-vapor. The residue was dissolved in ethyl acetate and washed with 1N HCl solution to remove DMAP. The organic layer was washed with water and dried over Na₂SO₄. Ethyl acetate evaporated and the residue was dried under high vacuum to afford 7-acetoxy-4-methylcoumarin. The acetylated coumarin was crystallized from petroleum ether-ethyl acetate mixture.

### A. Preparation of Platelet Rich, Plasma for the IN VITRO Studies

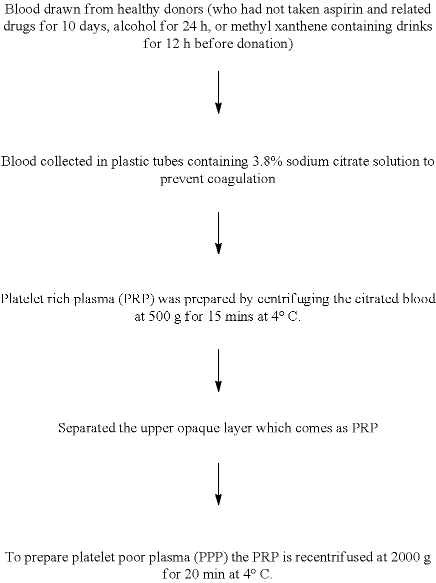

### B. Aggregation Studies

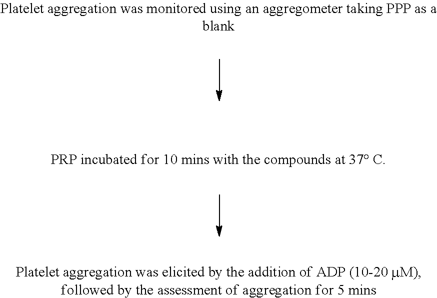

### C. Preparation of Platelet Rich Plasma for the EX VIVO Studies

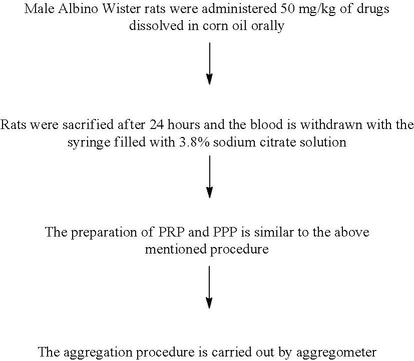

We claim:

1. A coumarin compound of formula I 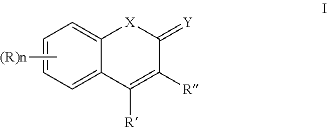
Where in—
X and Y represent O, S, NR′;
Rₙ represents one or several alkyl, aryl, OR₁, NH₂, SR₁, NR₁R₂
wherein R₁, R₂=H, alkyl, phenyl, aryl, OCOR₃, SCOR₃, NHCOR₃, NR₁COR₃, etc.
wherein R₃ represents alkyl, phenyl, aryl, heteroaryl.
R′ and R″ represent H, alkyl, phenyl, substituted phenyl, phenyloxy, substituted phenyloxy, amino, monosubstituted amino, disubstituted amino, aryl, heteroaryl, aryloxy, heteroaryloxy, alkoxy, thioalkyl, thioalkyloxy, halo, etc.

2. The coumarin compound as claimed in claim I, wherein X and Y are O.

3. The coumarin compound as claimed in claim I, wherein (R)n is selected from one or two OH, OCO-alkyl, OCO-aryl or O-alkyl.

4. The coumarin compound as claimed in claim I, wherein R′ and R″ are selected from alkyl, phenyl, aryl or heteroaryl.

5. A process for the preparation of compound of formula I comprising the steps of:
mixing polyphosphoric acid, a phenol and ethyl acetoacetate in the ratio 3.25:1:1 at 70-90° C. for 15-30 minutes to obtain a coumarin;
acylating said coumarin to obtain the product as claimed in claim I;
Purifying the product obtained.

6. The process as claimed in claim 1 wherein the acylating agent is an acid anhydride.

7. The process as claimed in claim I, wherein the phenol is resorcinol.

8. A pharmaceutical formulation comprising therapeutically effective amount of the compound as claimed in claim I and any pharmaceutical excipient thereof.

9. The coumarin compound as claimed in claim 1, for use in the inhibition of platelet aggregation.

10. The coumarin compound as claimed in claim 1, for use in the treatment of cardiovascular diseases.
